(19) United States
(12) Patent Application Publication
(10) Pub. No.: US 2022/0392060 A1
(43) Pub. Date: Dec. 8, 2022

(54) System, Microscope System, Methods and Computer Programs for Training or Using a Machine-Learning Model

(71) Applicants: George THEMELIS, Lindau (DE); Leica Instruments (Singapore) Pte. Ltd., Singapore (SG)

(72) Inventor: George THEMELIS, Lindau (DE)

(21) Appl. No.: 17/755,718

(22) PCT Filed: Oct. 30, 2020
(86) PCT No.: PCT/EP2020/080486

§ 371 Date: May 6, 2022

(30) Foreign Application Priority Data
Nov. 8, 2019 (DE) 10 2019 130 218.8

(51) Int. Cl. G06T 7/00

(52) U.S. Cl. CPC G06T 7/0012; G06T 2207/20081; G06T 2207/30024; G06T 2207/10056; G06T 2207/10064

(57) ABSTRACT

Examples relate to a system, a method and a computer program for training a machine-learning model, to a machine-learning model, a method and computer program for detecting at least one property of a sample of organic tissue, and to a microscope system. The system comprises one or more storage modules and one or more processors. The system is configured to obtain a plurality of images of a sample of organic tissue. The plurality of images are taken using a plurality of different imaging characteristics. The system is configured to train a machine-learning model using the plurality of images. The plurality of images are used as training samples and information on at least one property of the sample of organic tissue is used as a desired output of the machine-learning model. The machine-learning model is trained such that the machine-learning model is suitable for detecting the at least one property of the sample of organic tissue in image input data reproducing (only) a proper subset of the plurality of different imaging characteristics. The system is configured to provide the machine-learning model.

### TECHNICAL FIELD

Examples relate to a system, a method and a computer program for training a machine-learning model, to a machine-learning model, a method and computer program for detecting at least one property of a sample of organic tissue, and to a microscope system.

### BACKGROUND

A major use of microscopes lies in the analysis of organic tissue. For example, a microscope may be used to gain a detailed view of organic tissue, enabling practitioners and surgeons to detect features of the tissue, such as pathologic (i.e. “unhealthy”) tissue among healthy organic tissue.

### SUMMARY

There may be a desire for an improved approach for an analysis of organic tissue, which enables a better detection of features of the tissue.

This desire is addressed by the subject-matter of the independent claims.

Embodiments of the present disclosure provide a system comprising one or more storage modules and one or more processors. The system is configured to obtain a plurality of images of a sample of organic tissue. The plurality of images are taken using a plurality of different imaging characteristics. The system is configured to train a machine-learning model using the plurality of images. The plurality of images are used as training samples and information on at least one property of the sample of organic tissue is used as a desired output of the machine-learning model. The machine-learning model is trained such that the machine-learning model is suitable for detecting the at least one property of the sample of organic tissue in image input data reproducing (only) a proper subset of the plurality of different imaging characteristics. The system is configured to provide the machine-learning model.

Certain features of organic tissue, such as the shape of features or such as pathologic tissue may be easier to detect in images being taken with different image characteristics. For example, at certain spectral bands, a reflectance, a fluorescence or a bioluminescence of parts of the organic tissue may be characteristic for pathologic tissue. By using multiple images of the same organic tissue, which have been taken using different image characteristics (e.g. at different spectral bands, at different imaging modes, using different polarizations etc.), the machine-learning model, which may provide a form of artificial intelligence, may be trained to deduce the occurrence of the at least one feature, such as the pathologic tissue, even from images, which only match a proper subset of the different image characteristics. For example, in addition to a white light reflectance image (color image) having the visible light spectrum or reflectance imaging as image characteristics, additional images may be used as training samples that have been taken at spectral bands where a property, such as pathologic tissue, stands out due to its reflectance or fluorescence. The machine-learning model may “learn” to detect the property using the input samples being taken using the plurality of imaging characteristics, so that, if input data that reproduces only a subset of the imaging characteristics is fed to the machine-learning model, a detection of the feature, such as pathologic or healthy tissue, is still feasible.

Embodiments of the present disclosure further provide a method for training a machine-learning model. The method comprises obtaining a plurality of images of a sample of organic tissue. The plurality of images are taken using a plurality of different imaging characteristics. The method comprises training a machine-learning model using the plurality of images. The plurality of images are used as training samples and information on at least one property of the sample of organic tissue is used as a desired output of the machine-learning model in the training of the machine-learning model. The machine-learning is trained such that the machine-learning model is suitable for detecting the at least one property of the sample of organic tissue in image input data reproducing a proper subset of the plurality of different imaging characteristics. The method comprises providing the machine-learning model. Embodiments of the present disclosure further provide a machine-learning model that is trained using the system or method.

Embodiments of the present disclosure further provide a method for detecting at least one property of the sample of organic tissue. The method comprises using the machine-learning model generated by the above system or method with image input data reproducing a proper subset of the plurality of different imaging characteristics.

Embodiments further provide a computer program with a program code for performing at least one of the above methods when the computer program is executed on a processor.

Such embodiments may be used in a microscope, such as a surgical microscope, e.g. to aid in the detection of the at least one feature during surgery. Embodiments of the present disclosure provide a microscope system comprising the above system or being configured to execute at least one of the methods.

### SHORT DESCRIPTION OF THE FIGURES

Some examples of apparatuses and/or methods will be described in the following by way of example only, and with reference to the accompanying figures, in which

FIG. 1 shows a block diagram of an embodiment of a system;

FIG. 2a shows a flow chart of an embodiment of a method for training a machine-learning model;

FIG. 2b shows a flow chart of an embodiment of a method for detecting at least one property of a sample of organic tissue;

FIG. 3 shows a block diagram of a microscope system; and

FIGS. 4a to 6b show schematic diagrams of a detection of at least one property of a sample of organic tissue.

### DETAILED DESCRIPTION

Various examples will now be described more fully with reference to the accompanying drawings in which some examples are illustrated. In the figures, the thicknesses of lines, layers and/or regions may be exaggerated for clarity.

Accordingly, while further examples are capable of various modifications and alternative forms, some particular examples thereof are shown in the figures and will subsequently be described in detail. However, this detailed description does not limit further examples to the particular forms described. Further examples may cover all modifications, equivalents, and alternatives falling within the scope of the disclosure. Same or like numbers refer to like or similar elements throughout the description of the figures, which may be implemented identically or in modified form when compared to one another while providing for the same or a similar functionality.

It will be understood that when an element is referred to as being “connected” or “coupled” to another element, the elements may be directly connected or coupled or via one or more intervening elements. If two elements A and B are combined using an “or”, this is to be understood to disclose all possible combinations, i.e. only A, only B as well as A and B, if not explicitly or implicitly defined otherwise. An alternative wording for the same combinations is “at least one of A and B” or “A and/or B”. The same applies, mutatis mutandis, for combinations of more than two Elements.

The terminology used herein for the purpose of describing particular examples is not intended to be limiting for further examples. Whenever a singular form such as “a,” “an” and “the” is used and using only a single element is neither explicitly or implicitly defined as being mandatory, further examples may also use plural elements to implement the same functionality. Likewise, when a functionality is subsequently described as being implemented using multiple elements, further examples may implement the same functionality using a single element or processing entity. It will be further understood that the terms “comprises,” “comprising,” “includes” and/or “including,” when used, specify the presence of the stated features, integers, steps, operations, processes, acts, elements and/or components, but do not preclude the presence or addition of one or more other features, integers, steps, operations, processes, acts, elements, components and/or any group thereof.

Unless otherwise defined, all terms (including technical and scientific terms) are used herein in their ordinary meaning of the art to which the examples belong.

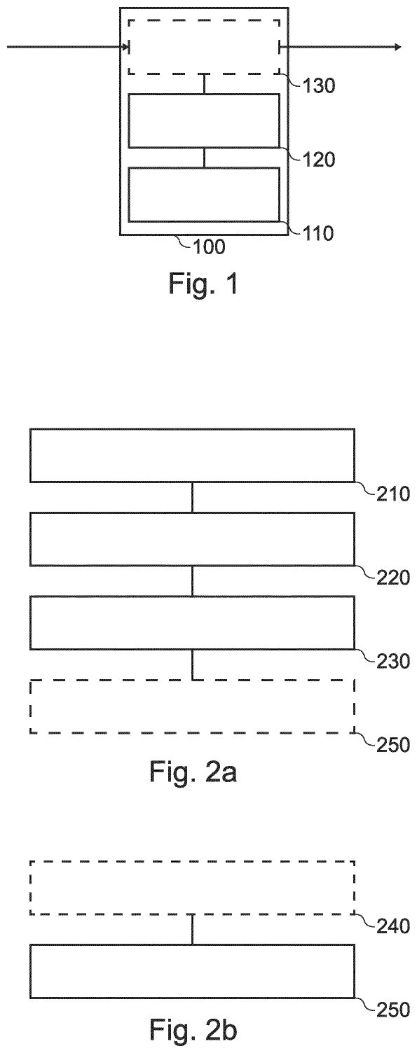

FIG. 1 shows a block diagram of an embodiment of a system 100 for training a machine-learning model. The system comprises one or more storage modules 110 and one or more processors 120, which is/are coupled to the one or more storage modules 110. Optionally, the system 100 comprises one or more interfaces 130, which may be coupled to the one or more processors 120, for obtaining and/or providing information, e.g. for providing the machine-learning model and/or for obtaining a plurality of images. In general, the one or more processors 120 of the system may be configured to execute the following tasks, e.g. in conjunction with the one or more storage modules 110 and/or the one or more interfaces 130.

The system is configured to obtain a plurality of images of a sample of organic tissue. The plurality of images are taken using a plurality of different imaging characteristics. The system is configured to train a machine-learning model using the plurality of images. The plurality of images are used as training samples. Information on at least one property of the sample of organic tissue is used as a desired output of the machine-learning model. The machine-learning model is trained such that the machine-learning model is suitable for detecting the at least one property of the sample of organic tissue in image input data reproducing (only) a proper subset of the plurality of different imaging characteristics (e.g. not the entire plurality of different imaging characteristics). The system is configured to provide the machine-learning model. For example, the system may be a computer-implemented system.

Embodiments provides a system, a method and a computer program for training a machine-learning model. Machine learning may refer to algorithms and statistical models that computer systems may use to perform a specific task without using explicit instructions, instead relying on models and inference. For example, in machine-learning, instead of a rule-based transformation of data, a transformation of data may be used, that is inferred from an analysis of historical and/or training data. For example, the content of images may be analyzed using a machine-learning model or using a machine-learning algorithm. In order for the machine-learning model to analyze the content of an image, the machine-learning model may be trained using training images as input and training content information as output. By training the machine-learning model with a large number of training images and/or training sequences (e.g. words or sentences) and associated training content information (e.g. labels or annotations), the machine-learning model “learns” to recognize the content of the images, so the content of images that are not included in the training data can be recognized using the machine-learning model.

Machine-learning models may be trained using training input data. The examples specified above use a training method called “supervised learning”. In supervised learning, the machine-learning model is trained using a plurality of training samples, wherein each sample may comprise a plurality of input data values, and a plurality of desired output values, i.e. each training sample is associated with a desired output value. By specifying both training samples and desired output values, the machine-learning model “learns” which output value to provide based on an input sample that is similar to the samples provided during the training.

In embodiments, this approach may be used on the plurality of images. In other words, the machine-learning model may be trained using supervised learning. The plurality of images are provided as training samples to the machine-learning model. For example, the plurality of images may be input at a plurality of inputs of the machine-learning model, e.g. at the same time. As a corresponding desired output, the information on the at least one property may be used. For example, the information on the at least one property of the sample of organic tissue may indicate at least one portion of the sample of organic tissue that is healthy or pathologic. Accordingly, information on pathologic or healthy tissue may be used as desired output of the training of the machine-learning model. In this case, the machine-learning model may be trained such that the machine-learning model is suitable for detecting pathologic or healthy tissue in image input data reproducing a proper subset of the plurality of different imaging characteristics. Alternatively or additionally, the information on the at least one property of the sample of organic tissue may indicate a shape of one or more features (e.g. blood vessels, distinct portions of the sample of organic tissue, bone structures etc.) of the sample of organic tissue. Accordingly, information on a shape of one or more features of the sample of organic tissue may be used as desired output of the training of the machine-learning model. In this case, the machine-learning model may be trained such that the machine-learning model is suitable for determining the shape of one or more features in image input data reproducing a proper subset of the plurality of different imaging characteristics. In general, the information on the at least one property of the sample of organic tissue, e.g. the information on the pathologic or healthy tissue or the information on the shape of the one or more features of the sample of organic tissue, may correspond to an image or bitmap, in which portions of the sample of organic tissue that indicate the at least one property of the sample of organic tissue are highlighted or denoted. To improve the machine-learning effort, the image or bitmap may have the same size, or at least the same aspect ratio, and/or represent the same segment of the sample of organic tissue, as the images of the plurality of images. To improve a precision of the training of the machine-learning model, thus improving the value gained from using images taken having a multitude of different image characteristics, the plurality of images may a) be precisely aligned with each other, so that a part of the organic tissue shown in a pixel in a first image is also shown in the corresponding pixel of a second image of the plurality of images, and b) the images may be taken substantially simultaneously, e.g. to make sure the organic tissue does not change between images. In other words, the system may be configured to correlate (i.e. precisely align) the plurality of images on a pixel-to-pixel basis. The machine-learning model may be trained based on the correlated plurality of images. Additionally or alternatively, the plurality of images may be substantially simultaneously-recorded images. In other words, the plurality of images may be taken within at most 30 seconds (or at most 15 seconds, at most 10 seconds, at most 5 seconds, at most 2 seconds, at most 1 second) from each other (applying to each pair of images of the plurality of images).

The system is configured to obtain a plurality of images of a sample of organic tissue. In biology, a tissue is an ensemble of similar cells (and an extracellular matrix), that have the same origin, and that together carry out a specific function. The term “organic” tissue denotes, that the tissue is part of or originates from an organic being, such as an animal, a human, or a plant. For example, the organic tissue may be (human) brain tissue, and the machine-learning model may be trained to detect brain tumors (the pathologic tissue being the brain tumor). For example, the plurality of images may be taken of the same sample of organic tissue, and from the same angle. The plurality of images may show the same segment of the sample of organic tissue. The plurality of images may be precisely aligned with each other, so that a part of the organic tissue shown in a pixel in a first image is also shown in the corresponding pixel of a second image of the plurality of images (e.g. after correlation of the plurality of images). In at least some examples, the plurality of images are a plurality of microscopic images, i.e. a plurality of images taken by a camera of a microscope.

The plurality of images are taken using a plurality of different imaging characteristics. In this context, the term “imaging characteristics” denotes that the plurality of images have been taken using different techniques resulting in images having different characteristics, albeit of the same organic tissue (at substantially the same time). For example, the plurality of images may be taken at different spectral bands, using different imaging modes (the imaging modes being at least two of reflectance imaging, fluorescence imaging and bioluminescence imaging), using different polarizations (e.g. circular polarization, linear polarization, linear polarization at different angles), and being different images of different points of time in a time-resolved imaging series. In other words, the plurality imaging characteristics may relate to at least one of different spectral bands, different imaging modes, different polarizations, and different points of time in a time-resolved imaging series. Accordingly, the plurality of images may comprise one or more elements of the group of microscopic images being taken at different spectral bands, microscopic images being taken at different imaging modes, microscopic images being taken with a different polarization, and microscopic images representing different points of time in a time-resolved imaging series.

Spectral images taken at different spectral bands may be images, in which a range of wavelengths (i.e. a “band”) of light being reproduced by the images is different. This can be reached by using different sensors (e.g. using sensors that are only sensitive to a certain range of wavelengths), different filters being placed in front of the sensors (the different filters filtering different ranges of wavelengths), or the sample of organic tissue being illuminated using different ranges of wavelengths of light.

By using different spectral bands, different imaging modes may be implemented, e.g. reflectance imaging, fluorescence imaging, or bioluminescence imaging. In reflectance imaging, light is reflected by the sample of organic tissue at the same wavelength(s) that is/are used to illuminate the sample of organic tissue, and the reflected light is reproduced by the respective images. In fluorescence imaging, light is emitted by the sample of organic tissue at a wavelength (or range of wavelengths) that is different from a wavelength (or range of wavelengths) used to illuminate the sample of organic tissue, and the emitted light is reproduced by the respective images. In bioluminescence imaging, the sample of tissue is not illuminated, but still emits light that is reproduced by the respective images. In reflectance imaging, fluorescence imaging, or bioluminescence imaging, one or more filters may be used to restrict a range of wavelengths being reproduced by the respective images. In the following, most examples relate to different spectral bands and/or different imaging modes being used.

In various embodiments, different spectral bands are used that are indicative of pathologic tissue, or that are used to detect fluorescent dyes that are applied to the sample of organic tissue. For example, fluorescein, indocyanine green (ICG) or 5-ALA (5-aminolevulinic acid) may be used as external fluorescent dyes. In other words, at least a subset of the images may be based on (either) the use of external fluorescent dyes or an autofluorescence of the sample of organic tissue. The fluorescent dye may be applied to a part of the sample of organic tissue that is pathologic, e.g. so it can be distinguished in at least one of the plurality of images taken at a corresponding spectral band. Additionally, in some cases, healthy or pathologic tissue, or certain features of the sample of organic tissue, may be auto-fluorescent, so it can (also) be distinguished in at least one of the plurality of images taken at a corresponding spectral band. Accordingly, at least a subset of the plurality of images may reproduce a spectral band that is tuned to at least one external fluorescent dye being applied to the sample of organic tissue, e.g. a spectral band at which light is emitted by the part of the organic tissue the fluorescent dye is applied to. Additionally or alternatively, at least a subset of the plurality of images reproduce a spectral band that is tuned to an auto-fluorescence of at least a part of the sample of organic tissue, e.g. a spectral band at which light is emitted by the part of the organic tissue that has auto-fluorescent properties.

In general, the plurality of images may comprise one or more reflectance spectral images and one or more fluorescence spectral images. For example, the one or more reflectance spectral images may reproduce the visible light spectrum. The one or more fluorescence spectral images may each reproduce a spectral band that is tuned to fluorescence at a specific wavelength being observable at the sample of organic tissue. Consequently, the plurality of images may comprise a subset of images in which the at least one property of the sample of organic tissue may be better distinguishable (i.e. the one or more fluorescence spectral images), and a further subset of images, that is likely to be used as input data in the detection of the at least one property. For example, the plurality of images may comprise a subset of images in which pathologic tissue may be better distinguishable from healthy tissue (i.e. the one or more fluorescence spectral images), and a further subset of images, that is likely to be used as input data in the detection of the healthy or pathologic tissue. Additionally or alternatively, the plurality of images may comprise a subset of images in which a shape of the one or more features is better distinguishable (i.e. the one or more fluorescence spectral images), and a further subset of images, that is likely to be used as input data in the detection of the shape of the one or more features.

Additionally or alternatively, different polarization may be used. Different polarizations may be used to restrict a direction or angle from which light incident to a camera is reproduced by the respective images. When using different polarizations, the plurality of images may comprise one or more elements of one or more images being taken without a polarization, one or more images being taken using a circular polarization, one or more images being taken using a linear polarization, and different images being taken at different angles of linear polarization.

In some embodiments, different time-resolved images may be used. For example, different images of different points of time in a time-resolved imaging series may be used for the plurality of images. In a time-resolved imaging series, a luminescence or fluorescence of the sample of organic tissue is recorded over a period of time (e.g. a second), as some luminescence or fluorescence effects take to appear, e.g. after illuminating the sample of organic tissue with light at a certain wavelength/band.

As has been pointed out above, the plurality of images comprises images taken using a plurality (e.g. at least 2, at least 3, at least 5, at least 8, at least 10) different imaging characteristics. So far, the images have been two-dimensional images. In other words, the plurality of images may be two-dimensional images.

In some cases, it may be beneficial to include three-dimensional data as well, as some pathologic tissue may be detectable due to its characteristic three-dimensional shape or surface structure. Accordingly, the plurality of images may comprise one or more three-dimensional representations of the sample of organic tissue. The one or more three-dimensional representations of the sample of organic tissue may comprise a three-dimensional surface representation of the sample of organic tissue and/or a (microscopic) imaging tomography-based three-dimensional representation of the sample of organic tissue. For example, the one or more three-dimensional representations of the sample of organic tissue may be precisely aligned with the two-dimensional images of the plurality of images. Additionally or alternatively, the one or more three-dimensional representations of the sample of organic tissue may show the same segment of the sample of organic tissue as the two-dimensional images of the plurality of images.

In various embodiments, an image of the plurality of images may be used as the desired output, or to determine the desired output, i.e. the information on the at least one property of the sample of organic tissue (e.g. the information on pathologic or healthy tissue or the information on a shape of one or more features), of the machine-learning model. This may enable a training of the machine-learning model without requiring human annotation of the image or images of the sample of organic tissue or without requiring to manually define the at least one property of the sample of organic tissue. In other words, the information on the at least one property of the sample of organic tissue may be based on an image, in the following denoted “reference image” (or “reference images”) of the plurality of images. Additionally or alternatively, the information on the at least one property of the sample of organic tissue may be based on the three-dimensional representation of the sample of organic tissue. The reference image may be processed to obtain (i.e. to determine or generate) the information on the at least one property of the sample of organic tissue. In other words, the system may be configured to process the image to obtain the information on the at least one property of the sample of organic tissue (e.g. the information on pathologic or healthy tissue or the information on a shape of one or more features).

Care may be taken in deciding which of the plurality of images to select as reference images. In general, an image may be selected in which the at least one property is clearly distinguishable or visible. In other words, the image may be taken using an imaging characteristic that is indicative for a specific property of the sample of organic tissue, e.g. that is indicative for a specific type of pathologic (or healthy) tissue or that is indicative for a shape of one or more features of the sample of organic tissue. As denoted earlier, fluorescence imaging may be used to obtain such images. In other words, the reference image may be a fluorescence spectral image, i.e. an image taken using fluorescence imaging. In such cases, the image may be excluded as a training sample, e.g. to avoid skewing the machine-learning model such, that it only works on input data being taken using the same imaging characteristic.

In some cases, multiple properties may be detected, e.g. multiple types of pathologic tissue or of multiple types of features might be present. In this case, for example, multiple reference images of the plurality of images may be used and/or processed to obtain the information on the at least one property of the sample of organic tissue. In other words, the information on the at least one property of the sample of organic tissue may be based on two or more (reference) images of the plurality of images. Each of the two or more images may be taken using an imaging characteristic that is indicative for a specific property of the sample of organic tissue, e.g. that is indicative for a specific type of pathologic (or healthy) tissue or that is indicative for a shape of one or more features of the sample of organic tissue. Consequently, the information on the at least one property may be based on a plurality of types of properties, e.g. based on a plurality of types of pathologic tissue or based on a plurality of types of features. In other words, in the information on the at least one property, a plurality of different types of properties of the sample of organic tissue may be highlighted or denoted, e.g. separately or in a combined fashion.

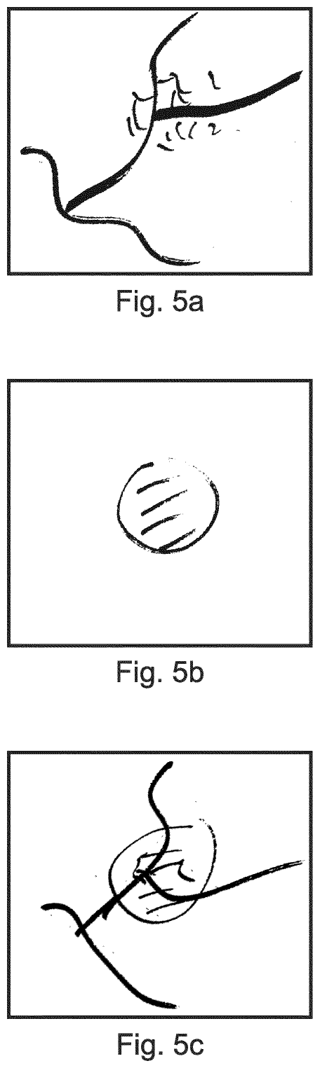

The machine-learning model is trained such, that the machine-learning model is suitable for detecting the at least one property of the sample of organic tissue in image input data reproducing a proper subset of the plurality of different imaging characteristics. In other words, the input data may cover (i.e. reproduce) fewer imaging characteristics than the plurality of images that is used as training samples of the machine-learning model. For example, the image input data may be image input data of a camera operating within the visible light spectrum, or image input data of a camera operating within the visible light spectrum, and that further comprises one, two or three additional reflectance images, fluorescence images or bioluminescence images. For example, the camera may be a camera of a microscope e.g. of the microscope 310 of FIG. 3. The machine-learning model may be trained such, that a detection of the at least one property yields (reliable) results if (only) a subset of the plurality of characteristics, such as only image input data of a camera operating within the visible light spectrum, is provided as input to the machine-learning model In some cases, the machine-learning model may be used in situations, in which fluorescent dyes cannot be used, for safety or cost reasons. Accordingly, the image input data may be taken of tissue not treated with an external fluorescent dye.

In various embodiments, a single sample of organic tissue might not be sufficient to properly train the machine-learning model. Accordingly, a plurality of samples of organic tissues may be used, along with a plurality of sets of a plurality of images. For example, each set of a plurality of images of the plurality of sets may be used together with a corresponding information on the at least one property of the sample of organic tissue, e.g. the machine-learning model may be trained with the plurality of images of a single set being applied at the inputs of the machine-learning model, and the corresponding information on the at least one property being used as the desired output of the machine-learning model.

As described above, the machine-learning model may be used to detect at least one property of the sample of organic tissue, e.g. healthy or pathologic tissue, or a shape of one or more features, in image input data. In other words, the system may be configured to use the machine-learning model with image input data reproducing the (proper) subset of the plurality of different imaging characteristics to detect the at least one property, e.g. to detect pathologic or healthy tissue, or the shape of one or more features, in the image input data. For example, the image input data may show or represent organic tissue, e.g. another sample of organic tissue (being different from the previously described sample of organic tissue.

In at least some embodiments, the system may be configured to overlay the image input data with a visual overlay indicating the at least one property of the sample of organic tissue, e.g. by highlighting pathologic or healthy tissue, or by highlighting a shape of the one or more features.

The one or more interfaces 130 may correspond to one or more inputs and/or outputs for receiving and/or transmitting information, which may be in digital (bit) values according to a specified code, within a module, between modules or between modules of different entities. For example, the one or more interfaces 130 may comprise interface circuitry configured to receive and/or transmit information.

In embodiments the one or more processors 120 may be implemented using one or more processing units, one or more processing devices, any means for processing, such as a processor, a computer or a programmable hardware component being operable with accordingly adapted software. In other words, the described function of the one or more processors 120 may as well be implemented in software, which is then executed on one or more programmable hardware components. Such hardware components may comprise a general-purpose processor, a Digital Signal Processor (DSP), a micro-controller, etc.

In at least some embodiments, the one or more storage modules 110 may comprise at least one element of the group of a computer readable storage medium, such as an magnetic or optical storage medium, e.g. a hard disk drive, a flash memory, Floppy-Disk, Random Access Memory (RAM), Programmable Read Only Memory (PROM), Erasable Programmable Read Only Memory (EPROM), an Electronically Erasable Programmable Read Only Memory (EEPROM), or a network storage.

More details and aspects of embodiments are mentioned in connection with the proposed concept or one or more examples described above or below. Embodiments may comprise one or more additional optional features corresponding to one or more aspects of the proposed concept or one or more examples described above or below.

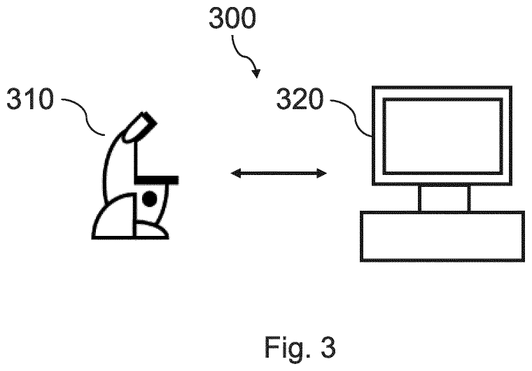

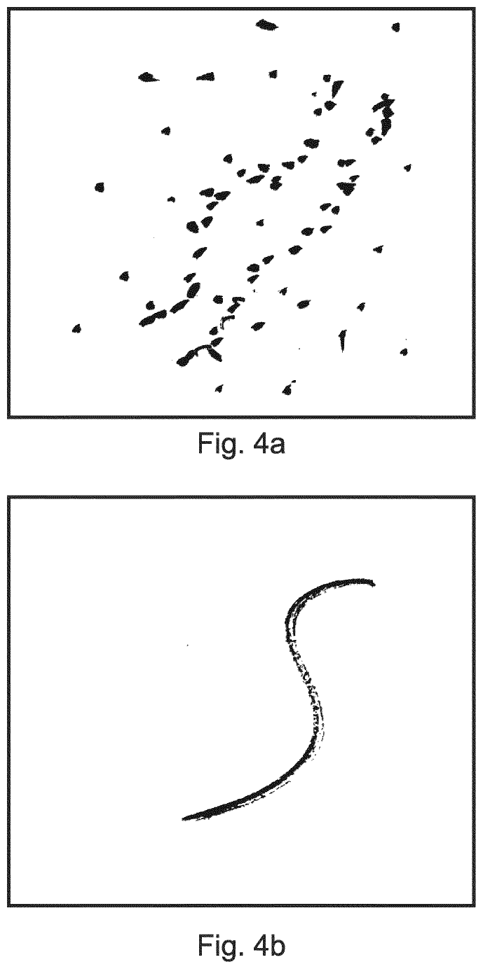

FIG. 2a shows a flow chart of an embodiment of a corresponding (computer-implemented) method for training a machine-learning model. The method comprises obtaining 210 a plurality of images of a sample of organic tissue. The plurality of images are taken using a plurality of different imaging characteristics. The method comprises training 220 a machine-learning model using the plurality of images. The plurality of images being used as training samples and information on at least one property of the sample of organic tissue are used as a desired output of the machine-learning model. The machine-learning model is trained such that the machine-learning model is suitable for detecting the at least one property of the sample of organic tissue in image input data reproducing a proper subset of the plurality of different imaging characteristics. The method comprises providing 230 the machine-learning model. Optionally, the method comprises using 250 the machine-learning model with image input data reproducing a proper subset of the plurality of different imaging characteristics to detect the at least one property of the sample of organic tissue.

Alternatively, the detecting may be performed separately from the training of the machine-learning model. Accordingly, the machine-learning model may be used within a microscope system, while the training is performed in a separate computer system. Accordingly, FIG. 2b shows a flow chart of an embodiment of a method for detecting at least one property of a sample of organic tissue. Optionally, the method comprises obtaining 240 the machine-learning model and/or image input data reproducing a proper subset of the plurality of different imaging characteristics. The method comprises using 250 the machine-learning model with image the input data reproducing a proper subset of the plurality of different imaging characteristics, e.g. to detect the at least one property of the sample of organic tissue.

More details and aspects of embodiments are mentioned in connection with the proposed concept or one or more examples described above or below. Embodiments may comprise one or more additional optional features corresponding to one or more aspects of the proposed concept or one or more examples described above or below.

FIG. 3 shows a block diagram of a microscope system 300. For example, the microscope system may be configured at least one of the methods of FIGS. 2a and/or 2b, and/or comprise the system of FIG. 1. Accordingly, some embodiments relate to a microscope comprising a system as described in connection with one or more of the FIGS. 1 to 2b. Alternatively, a microscope may be part of or connected to a system as described in connection with one or more of the FIGS. 1 to 2b. FIG. 3 shows a schematic illustration of a microscope system 300 configured to perform a method described herein. The system 300 comprises a microscope 310 and a computer system 320. The microscope 310 is configured to take images and is connected to the computer system 320. The computer system 320 is configured to execute at least a part of a method described herein. The computer system 320 may be configured to execute a machine learning algorithm. The computer system 320 and microscope 310 may be separate entities but can also be integrated together in one common housing. The computer system 320 may be part of a central processing system of the microscope 310 and/or the computer system 320 may be part of a subcomponent of the microscope 310, such as a sensor, an actor, a camera or an illumination unit, etc. of the microscope 310.

The computer system 320 may be a local computer device (e.g. personal computer, laptop, tablet computer or mobile phone) with one or more processors and one or more storage devices or may be a distributed computer system (e.g. a cloud computing system with one or more processors and one or more storage devices distributed at various locations, for example, at a local client and/or one or more remote server farms and/or data centers). The computer system 320 may comprise any circuit or combination of circuits. In one embodiment, the computer system 320 may include one or more processors which can be of any type. As used herein, processor may mean any type of computational circuit, such as but not limited to a microprocessor, a microcontroller, a complex instruction set computing (CISC) microprocessor, a reduced instruction set computing (RISC) microprocessor, a very long instruction word (VLIW) microprocessor, a graphics processor, a digital signal processor (DSP), multiple core processor, a field programmable gate array (FPGA), for example, of a microscope or a microscope component (e.g. camera) or any other type of processor or processing circuit. Other types of circuits that may be included in the computer system 320 may be a custom circuit, an application-specific integrated circuit (ASIC), or the like, such as, for example, one or more circuits (such as a communication circuit) for use in wireless devices like mobile telephones, tablet computers, laptop computers, two-way radios, and similar electronic systems. The computer system 320 may include one or more storage devices, which may include one or more memory elements suitable to the particular application, such as a main memory in the form of random access memory (RAM), one or more hard drives, and/or one or more drives that handle removable media such as compact disks (CD), flash memory cards, digital video disk (DVD), and the like. The computer system 320 may also include a display device, one or more speakers, and a keyboard and/or controller, which can include a mouse, trackball, touch screen, voice-recognition device, or any other device that permits a system user to input information into and receive information from the computer system 320.

Some or all of the method steps may be executed by (or using) a hardware apparatus, like for example, a processor, a microprocessor, a programmable computer or an electronic circuit. In some embodiments, some one or more of the most important method steps may be executed by such an apparatus.

Depending on certain implementation requirements, embodiments of the invention can be implemented in hardware or in software. The implementation can be performed using a non-transitory storage medium such as a digital storage medium, for example a floppy disc, a DVD, a Blu-Ray, a CD, a ROM, a PROM, and EPROM, an EEPROM or a FLASH memory, having electronically readable control signals stored thereon, which cooperate (or are capable of cooperating) with a programmable computer system such that the respective method is performed. Therefore, the digital storage medium may be computer readable.

Some embodiments according to the invention comprise a data carrier having electronically readable control signals, which are capable of cooperating with a programmable computer system, such that one of the methods described herein is performed.

Generally, embodiments of the present invention can be implemented as a computer program product with a program code, the program code being operative for performing one of the methods when the computer program product runs on a computer. The program code may, for example, be stored on a machine readable carrier.

Other embodiments comprise the computer program for performing one of the methods described herein, stored on a machine readable carrier.

In other words, an embodiment of the present invention is, therefore, a computer program having a program code for performing one of the methods described herein, when the computer program runs on a computer.

A further embodiment of the present invention is, therefore, a storage medium (or a data carrier, or a computer-readable medium) comprising, stored thereon, the computer program for performing one of the methods described herein when it is performed by a processor. The data carrier, the digital storage medium or the recorded medium are typically tangible and/or non-transitionary. A further embodiment of the present invention is an apparatus as described herein comprising a processor and the storage medium.

A further embodiment of the invention is, therefore, a data stream or a sequence of signals representing the computer program for performing one of the methods described herein. The data stream or the sequence of signals may, for example, be configured to be transferred via a data communication connection, for example, via the internet.

A further embodiment comprises a processing means, for example, a computer or a programmable logic device, configured to, or adapted to, perform one of the methods described herein.

A further embodiment comprises a computer having installed thereon the computer program for performing one of the methods described herein.

A further embodiment according to the invention comprises an apparatus or a system configured to transfer (for example, electronically or optically) a computer program for performing one of the methods described herein to a receiver. The receiver may, for example, be a computer, a mobile device, a memory device or the like. The apparatus or system may, for example, comprise a file server for transferring the computer program to the receiver.

In some embodiments, a programmable logic device (for example, a field programmable gate array) may be used to perform some or all of the functionalities of the methods described herein. In some embodiments, a field programmable gate array may cooperate with a microprocessor in order to perform one of the methods described herein. Generally, the methods are preferably performed by any hardware apparatus.

More details and aspects of embodiments are mentioned in connection with the proposed concept or one or more examples described above or below. Embodiments may comprise one or more additional optional features corresponding to one or more aspects of the proposed concept or one or more examples described above or below.

At least some embodiments relate to a use of Artificial Intelligence (AI), e.g. in the form of the machine-learning model, in a microscope, e.g. to interpret the images captured with the surgical microscope. For example, (all) available information may be collected and the AI may be used to increase diagnostic information. In embodiments, artificial intelligence is used in surgical microscopes to interpret the images captured.

The limitation of some microscope systems, if they are to be used with AI, is the collection of images, both for training and applying the AI. Specifically, the microscopes acquire the different images sequentially, and therefore it may be more difficult and cumbersome to collect the different data. Even more difficult may be the step of data annotation, i.e. employing an expert surgeon to annotate the healthy and pathologic images, or even more difficult to manually segment the healthy and diseased tissue areas.

The inventor proposes a way to do the training and application of AI in surgical microscope easier, more efficient, and more accurate. In at least some embodiments, the microscope is able to capture multiple images of reflectance and fluorescence (e.g. the plurality of images) simultaneously and in real time (e.g. substantially simultaneously). In other words, for each frame, the camera captures multiple images, in some examples up to 3 reflectance and 3 fluorescence spectral images. This number may increase in the future. The ability to capture the images simultaneous and instantaneous allows to correlate the images on a pixel to pixel basis. This plurality of images, which can be correlated pixel to pixel, offer a great platform for AI, i.e. for training a machine-learning model, since more data may be available for neural network correlation. Consequently, embodiments may be based on using a plurality of images captured at different spectral bands, in reflectance and fluorescence, with the use of external fluorescent dyes such as fluorescein, indocyanine green (ICG) and 5-ALA, or tissue autofluorescence (without fluorescent dye) and try to train the system to detect different pathologic and/or healthy tissue. A specific case where the use of AI could be relatively easy and non-obvious is the use of 5-ALA induced fluorescence images to train the system (i.e. the machine-learning model) to detect brain tumor from non-fluorescence images. Specifically, 5-ALA causes brain tumor tissue to emit fluorescence, with relatively good sensitivity and specificity, and is thus used in intraoperative guidance for brain tumor excision. In other words, from the fluorescence image it is fairly easy to segment the tumor areas, just by setting a fluorescence intensity threshold.

This can be done fully automatically by a computer, and thereby automatically annotate the captured images (e.g. to obtain the information on pathologic or healthy tissue) without the necessity of human intervention, even though the review by an expert may add security and confidence. The ability of the system to capture simultaneously the white light reflectance image (color image) allows to collect data in real time during the whole duration of such surgical operations. This may eliminate the time consuming and expensive process of capturing and annotating the images. The goal of this AI/machine-learning training would be to try to guess the presence of tumor in the brain just by looking at the tissue without administering 5-ALA which is expensive and not always availably due to financial or regulatory reasons.

More details and aspects of embodiments are mentioned in connection with the proposed concept or one or more examples described above or below. Embodiments may comprise one or more additional optional features corresponding to one or more aspects of the proposed concept or one or more examples described above or below.

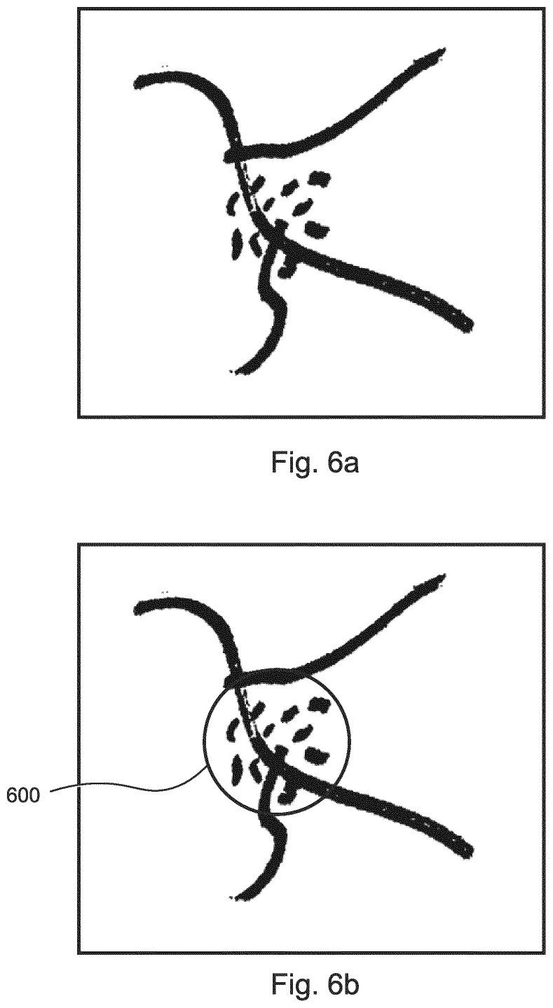

FIGS. 4a to 6b show schematic diagrams of a detection of at least one property of a sample of organic tissue. In FIGS. 4a and 4b, a sample of organic tissue is shown, where a shape of a feature of the sample of organic tissue is visible, but hard to clearly make out in a first image (FIG. 4a) being taken with a first imaging characteristic (e.g. white light reflectance imaging), but clearly visible in a second image (FIG. 4b) being taken with a second imaging characteristic (e.g. using fluorescence imaging, with the feature of the sample of organic tissue being infused with a fluorescent dye). In such cases, the second image may be used to determine the shape of the feature of the sample of organic tissue, and may be used to generate the desired output for the training of the machine-learning model, so that the machine-learning model can be used to determine the shape of the feature using the first image alone.

In FIGS. 5a to 5c, a similar scenario is shown. This time, two distinct features (blood vessels, which are shown as lines, and a portion of a tissue having a certain property, shown as dots) are visible within a first image (FIG. 5a), which is taken with a first imaging characteristic (e.g. white light reflectance imaging). In a second image (FIG. 5b), the shape of the portion of the tissue is clearly visible. For example, the second image may be taken using fluorescence imaging. Thus, the second image may be used to generate the information on the at least one property of the sample of organic tissue, and to train the machine-learning model accordingly, such that the machine-learning model is suitable for identifying the shape of the portion of the sample of organic tissue from the first image alone, and to superimpose the shape on the first image to generate a third image (see FIG. 5c).

A similar example is shown in FIGS. 6a and 6b. By using the machine-learning model (i.e. artificial intelligence), the first image (FIG. 6a), which may be a white-light reflectance imaging being taken by a camera may be annotated to show (FIG. 6a) the shape 600 of a region of the sample of organic tissue.

More details and aspects of embodiments are mentioned in connection with the proposed concept or one or more examples described above or below (e.g. FIGS. 1 to 3). Embodiments may comprise one or more additional optional features corresponding to one or more aspects of the proposed concept or one or more examples described above or below.

Embodiments may be based on using a machine-learning model or machine-learning algorithm. Machine learning may refer to algorithms and statistical models that computer systems may use to perform a specific task without using explicit instructions, instead relying on models and inference. For example, in machine-learning, instead of a rule-based transformation of data, a transformation of data may be used, that is inferred from an analysis of historical and/or training data. For example, the content of images may be analyzed using a machine-learning model or using a machine-learning algorithm. In order for the machine-learning model to analyze the content of an image, the machine-learning model may be trained using training images as input and training content information as output. By training the machine-learning model with a large number of training images and/or training sequences (e.g. words or sentences) and associated training content information (e.g. labels or annotations), the machine-learning model “learns” to recognize the content of the images, so the content of images that are not included in the training data can be recognized using the machine-learning model. The same principle may be used for other kinds of sensor data as well: By training a machine-learning model using training sensor data and a desired output, the machine-learning model “learns” a transformation between the sensor data and the output, which can be used to provide an output based on non-training sensor data provided to the machine-learning model. The provided data (e.g. sensor data, meta data and/or image data) may be preprocessed to obtain a feature vector, which is used as input to the machine-learning model.

Machine-learning models may be trained using training input data. The examples specified above use a training method called “supervised learning”. In supervised learning, the machine-learning model is trained using a plurality of training samples, wherein each sample may comprise a plurality of input data values, and a plurality of desired output values, i.e. each training sample is associated with a desired output value. By specifying both training samples and desired output values, the machine-learning model “learns” which output value to provide based on an input sample that is similar to the samples provided during the training. Apart from supervised learning, semi-supervised learning may be used. In semi-supervised learning, some of the training samples lack a corresponding desired output value. Supervised learning may be based on a supervised learning algorithm (e.g. a classification algorithm, a regression algorithm or a similarity learning algorithm. Classification algorithms may be used when the outputs are restricted to a limited set of values (categorical variables), i.e. the input is classified to one of the limited set of values. Regression algorithms may be used when the outputs may have any numerical value (within a range). Similarity learning algorithms may be similar to both classification and regression algorithms but are based on learning from examples using a similarity function that measures how similar or related two objects are. Apart from supervised or semi-supervised learning, unsupervised learning may be used to train the machine-learning model. In unsupervised learning, (only) input data might be supplied and an unsupervised learning algorithm may be used to find structure in the input data (e.g. by grouping or clustering the input data, finding commonalities in the data). Clustering is the assignment of input data comprising a plurality of input values into subsets (clusters) so that input values within the same cluster are similar according to one or more (pre-defined) similarity criteria, while being dissimilar to input values that are included in other clusters.

Reinforcement learning is a third group of machine-learning algorithms. In other words, reinforcement learning may be used to train the machine-learning model. In reinforcement learning, one or more software actors (called “software agents”) are trained to take actions in an environment. Based on the taken actions, a reward is calculated. Reinforcement learning is based on training the one or more software agents to choose the actions such, that the cumulative reward is increased, leading to software agents that become better at the task they are given (as evidenced by increasing rewards).

Furthermore, some techniques may be applied to some of the machine-learning algorithms. For example, feature learning may be used. In other words, the machine-learning model may at least partially be trained using feature learning, and/or the machine-learning algorithm may comprise a feature learning component. Feature learning algorithms, which may be called representation learning algorithms, may preserve the information in their input but also transform it in a way that makes it useful, often as a pre-processing step before performing classification or predictions. Feature learning may be based on principal components analysis or cluster analysis, for example.

In some examples, anomaly detection (i.e. outlier detection) may be used, which is aimed at providing an identification of input values that raise suspicions by differing significantly from the majority of input or training data. In other words, the machine-learning model may at least partially be trained using anomaly detection, and/or the machine-learning algorithm may comprise an anomaly detection component.

In some examples, the machine-learning algorithm may use a decision tree as a predictive model. In other words, the machine-learning model may be based on a decision tree. In a decision tree, observations about an item (e.g. a set of input values) may be represented by the branches of the decision tree, and an output value corresponding to the item may be represented by the leaves of the decision tree. Decision trees may support both discrete values and continuous values as output values. If discrete values are used, the decision tree may be denoted a classification tree, if continuous values are used, the decision tree may be denoted a regression tree.

Association rules are a further technique that may be used in machine-learning algorithms. In other words, the machine-learning model may be based on one or more association rules.

Association rules are created by identifying relationships between variables in large amounts of data. The machine-learning algorithm may identify and/or utilize one or more relational rules that represent the knowledge that is derived from the data. The rules may e.g. be used to store, manipulate or apply the knowledge.

Machine-learning algorithms are usually based on a machine-learning model. In other words, the term “machine-learning algorithm” may denote a set of instructions that may be used to create, train or use a machine-learning model. The term “machine-learning model” may denote a data structure and/or set of rules that represents the learned knowledge (e.g. based on the training performed by the machine-learning algorithm). In embodiments, the usage of a machine-learning algorithm may imply the usage of an underlying machine-learning model (or of a plurality of underlying machine-learning models). The usage of a machine-learning model may imply that the machine-learning model and/or the data structure/set of rules that is the machine-learning model is trained by a machine-learning algorithm.

For example, the machine-learning model may be an artificial neural network (ANN). ANNs are systems that are inspired by biological neural networks, such as can be found in a retina or a brain. ANNs comprise a plurality of interconnected nodes and a plurality of connections, so-called edges, between the nodes. There are usually three types of nodes, input nodes that receiving input values, hidden nodes that are (only) connected to other nodes, and output nodes that provide output values. Each node may represent an artificial neuron. Each edge may transmit information, from one node to another. The output of a node may be defined as a (non-linear) function of its inputs (e.g. of the sum of its inputs). The inputs of a node may be used in the function based on a “weight” of the edge or of the node that provides the input. The weight of nodes and/or of edges may be adjusted in the learning process. In other words, the training of an artificial neural network may comprise adjusting the weights of the nodes and/or edges of the artificial neural network, i.e. to achieve a desired output for a given input.

Alternatively, the machine-learning model may be a support vector machine, a random forest model or a gradient boosting model. Support vector machines (i.e. support vector networks) are supervised learning models with associated learning algorithms that may be used to analyze data (e.g. in classification or regression analysis). Support vector machines may be trained by providing an input with a plurality of training input values that belong to one of two categories. The support vector machine may be trained to assign a new input value to one of the two categories. Alternatively, the machine-learning model may be a Bayesian network, which is a probabilistic directed acyclic graphical model. A Bayesian network may represent a set of random variables and their conditional dependencies using a directed acyclic graph. Alternatively, the machine-learning model may be based on a genetic algorithm, which is a search algorithm and heuristic technique that mimics the process of natural selection.

As used herein the term “and/or” includes any and all combinations of one or more of the associated listed items and may be abbreviated as “/”.

Although some aspects have been described in the context of an apparatus, it is clear that these aspects also represent a description of the corresponding method, where a block or device corresponds to a method step or a feature of a method step. Analogously, aspects described in the context of a method step also represent a description of a corresponding block or item or feature of a corresponding apparatus. Some or all of the method steps may be executed by (or using) a hardware apparatus, like for example, a processor, a microprocessor, a programmable computer or an electronic circuit. In some embodiments, some one or more of the most important method steps may be executed by such an apparatus.

### LIST OF REFERENCE SIGNS

100 System
110 One or more storage modules
120 One or more processors
130 One or more interfaces
210 Obtaining a plurality of images of a sample of organic tissue
220 Training a machine-learning model
230 Providing the machine-learning model
240 Obtaining a machine-learning model
250 Using the machine-learning model
300 Microscope system
310 Microscope
320 Computer system
600 Region of a sample of organic tissue

1. A system comprising one or more storage modules and one or more processors, wherein the system is configured to:
obtain a plurality of images of a sample of organic tissue, the plurality of images being taken using a plurality of different imaging characteristics;
train a machine-learning model using the plurality of images, the plurality of images being used as training samples and information on at least one property of the sample of organic tissue being used as a desired output of the machine-learning model, such that the machine-learning model is suitable for detecting the at least one property of the sample of organic tissue in image input data reproducing a proper subset of the plurality of different imaging characteristics; and
provide the machine-learning model.

2. The system according to claim 1, wherein the information on the at least one property of the sample of organic tissue indicates at least one portion of the sample of organic tissue that is healthy or pathologic, and/or wherein the information on the at least one property of the sample of organic tissue indicates a shape of one or more features of the sample of organic tissue.

3. The system according to claim 1, wherein information on pathologic or healthy tissue is used as desired output of the training of the machine-learning model, wherein the machine-learning model is trained such that the machine-learning model is suitable for detecting pathologic or healthy tissue in image input data reproducing a proper subset of the plurality of different imaging characteristics.

4. The system according to claim 1, wherein information on a shape of one or more features of the sample of organic tissue is used as desired output of the training of the machine-learning model, wherein the machine-learning model is trained such that the machine-learning model is suitable for determining the shape of one or more features in image input data reproducing a proper subset of the plurality of different imaging characteristics.

5. The system according to claim 1, wherein the plurality of different imaging characteristics relate to at least one of different spectral bands, different imaging modes, different polarizations, and different points of time in a time-resolved imaging series.

6. The system according to claim 1, wherein the plurality of images comprise one or more elements of the group of microscopic images being taken at different spectral bands, microscopic images being taken at different imaging modes, microscopic images being taken with a different polarization, and microscopic images representing different points of time in a time-resolved imaging series.

7. The system according to claim 1, wherein the plurality of images comprise one or more three-dimensional representations of the sample of organic tissue, and/or wherein the information on the at least one property of the sample of organic tissue is based on the three-dimensional representation of the sample of organic tissue.

8. The system according to claim 1, wherein the information on the at least one property of the sample of organic tissue is based on an image of the plurality of images, wherein the system is configured to process the image to obtain the information on the at least one property of the sample of organic tissue.

9. The system according to claim 8, wherein the image is taken using an imaging characteristic that is indicative for a specific type of pathologic tissue, and/or wherein the image is taken using an imaging characteristic that is indicative for a shape of one or more features of the sample of organic tissue, and/or wherein the image is a fluorescence spectral image, and/or wherein the image is excluded as training sample.

10. The system according to claim 1, wherein the information on the at least one property of the sample of organic tissue is based on two or more images of the plurality of images, wherein each of the two or more images are taken using an imaging characteristic that is indicative for a specific type of pathologic tissue, or wherein each of the two or more images are taken using an imaging characteristic that is indicative for a shape of one or more features of the sample of organic tissue.

11. The system according to claim 1, wherein at least a subset of the plurality of images reproduce a spectral band that is tuned to at least one external fluorescent dye being applied to the sample of organic tissue, and/or wherein at least a subset of the plurality of images reproduce a spectral band that is tuned to an autofluorescence of at least a part of the sample of organic tissue.

12. The system according to claim 1, wherein the system is configured to correlate the plurality of images on a pixel-to-pixel basis, wherein the machine-learning model is trained based on the correlated plurality of images.

13. The system according to claim 1, wherein the plurality of images comprise one or more reflectance spectral images and one or more fluorescence spectral images, and wherein the one or more reflectance spectral images reproduce the visible light spectrum and/or wherein the one or more fluorescence spectral images each reproduce a spectral band that is tuned to fluorescence at a specific wavelength being observable at the sample of organic tissue.

14. The system according to claim 1, wherein the system is configured to use the machine-learning model with image input data reproducing the proper subset of the plurality of different imaging characteristics to detect the at least one property of the sample of organic tissue in the image input data.

15. The system according to claim 14, wherein the image input data is image input data of a camera operating within the visible light spectrum, and/or wherein the image input data is taken of tissue not treated with an external fluorescent dye.

16. (canceled)

17. A method for training a machine-learning model, the method comprising:
obtaining a plurality of images of a sample of organic tissue, the plurality of images being taken using a plurality of different imaging characteristics;
training a machine-learning model using the plurality of images, the plurality of images being used as training samples and information on at least one property of the sample of organic tissue being used as a desired output of the machine-learning model, such that the machine-learning model is suitable for detecting the at least one property of the sample of organic tissue in image input data reproducing a proper subset of the plurality of different imaging characteristics; and
providing the machine-learning model.

18. A microscope system configured to detect at least one property of a sample of organic tissue, the microscope system being configured to use a machine-learning model of claim 16 with image input data reproducing a proper subset of the plurality of different imaging characteristics.

19. A non-transitory, computer-readable medium with a program code for performing the method according to claim 17, when the program code is executed on a processor.

20. (canceled)
